(19) United States
(12) Patent Application Publication
(10) Pub. No.: US 2010/0098734 A1
(43) Pub. Date: Apr. 22, 2010

(54) METHODS FOR PREPARATION OF A THIXOTROPIC MICROEMULSION FOR SKIN CARE FORMULATIONS

(76) Inventor: JOHN JACOB WILLE, JR., Chesterfield, NJ (US)

Correspondence Address: William H. Dippert;Eckert Seamans Cherin & Mellott, LLC, U.S. Steel Tower, 600 Grant Street, 44th Floor, Pittsburgh, PA, 15219, US

(21) Appl. No.: 12/619,982

(22) Filed: Nov. 17, 2009

Related U.S. Application Data
(63) Continuation-in-part of application No. 10/873,590, filed on Jun. 22, 2004.
Application No. 10/873,590 is a continuation-in-part of application No. 11/339,419, filed on Jan. 25, 2006.
(60) Provisional application No. 60/485,058, filed on Jul. 7, 2003.
(60) Provisional application No. 60/646,896, filed on Jan. 25, 2005.

(51) Int. Cl. A61K 8/02; A61K 9/113

(52) U.S. Cl. 424/401; 424/484; 424/488; 424/405

(57) ABSTRACT

Methods for the preparation of a novel topical gel delivery system are disclosed. The delivery system is an oil-in-water (O/W) type thixotropic microemulsion especially useful as a vehicle for the delivery of botanical actives. The delivery system is comprised of natural starches emulsified with a cationic surfactant and utilizes both synthetic and cosmetically-acceptable oils in a two step process. The resulting microemulsion is a uniform dispersion of oil droplets in a stable starch-oil composite. The method also allows for sequestering volatile fragrances by encapsulating them in the oil phase droplets, drying of liquid emulsions to a thin film, and subsequent moisture-activated release of the entrapped fragrances from dried films. Finally, a wound dressing that undergoes reversible hydration upon contact with wound exudates can be prepared by the methods of this invention.

### CROSS-REFERENCE TO RELATED APPLICATIONS

This application claims priority from Provisional Patent Application 60/485,058 filed Jul. 7, 2003, and its corresponding utility patent application Ser. No. 10/873,590 filed Jun. 22, 2004, entitled “Novel Topical Delivery System for Plant Derived Anti-irritants.” This application also claims priority from Provisional Application 60/646,896 filed Jan. 25, 2005, and its corresponding utility patent application Ser. No. 11/339,419 filed Jan. 25, 2006, entitled “Moisture-Activated Release of fragrances from a Novel Pourable Lotion Formulations.” This application is a consolidation and continuation-in-part of the aforesaid applications.

### TECHNICAL FIELD OF THE INVENTION

The field of the invention is in cosmetic and personal care formulations and in the topical delivery of botanical actives, fragrances, and oxygen carrier gels.

### BACKGROUND OF THE INVENTION

The creation of cosmetic and personal care product formulations containing multiple ingredients presents many difficulties and challenges due to unanticipated behavior of any particular ingredient in the final formulation. One problem confronting formulators of fragrances is their instability upon prolonged product storage and or conversion to less stable forms during thermal processing of the final product formulation. Another concern is the interaction of stabilizing fixatives with other formulation ingredients. A still further concern is to provide for controlled and slow release of the desired fragrance after application to the desired body location. The unique and unobvious methods and formulations disclosed in this patent application deal with the above cited difficulties in formulating a stable and temporally-controlled release of fragrances through the application of a novel starch encapsulation technology.

Encapsulation technology has received considerable attention for high volume applications such as household products, personal care, agriculture, packaging and coatings (Pothakamury U R and Barboa-Canovas G V. 1995. Trend in Food science & Technology 6: 397-406), including laundry products, cosmetics CR (controlled release) vitamins, CR probiotics, CR agrochemicals, CR plant hormones and antifungal compounds.

Environmental concerns over the large-scale use of solvents and cost associated with conventional matrix forming technologies such as solvent evaporation, emulsion encapsulants have prevented the use of these technologies for high volume applications. The objective of cost-effectiveness and efficiency can be accomplished by application of continuous processing techniques using an abundant matrix material to obtain encapsulation with tailored release properties. Starch is a widely employed matrix material. It possesses many favorable properties including abundance, low cost, process ability, biodegradability and ease of chemical and enzymatic modification. It is capable of extrusion processing and encapsulation can be accomplished with heat sensitive compounds.

Starch-based delivery systems have been recently reviewed (Freers S O, “Starch-based delivery systems, in Meyer R. Rosen (ed), Delivery System Handbook for Personal Care and Cosmetic Products, 741-760, 2005 William Andrew, Inc). In this review, the author discuss the various chemical and physical modification of starch that allow formulators to lessen retrograde gelation, attain controlled hydration, and means to lower the gelation temperature thus providing formulators ease and flexibility in processing conditions. Acid and enzymatic modifications of starch_granules and chemical modification of starch granules have allowed cold processing, starch stabilization by radiation cross-linking, called “stabilized starches” have lowered gelation temperature and required less heat for hydration. Finally, starches can slightly oxidized by a variety of chemical oxidizing agents, including hydrogen peroxide, and sodium hypochlorite, which produce starches with lowered bioburden and improved adhesion at lower gelatinization temperatures.

Starch encapsulation of hydrophobic compounds was reported using a novel starch based matrix for encapsulation of heat sensitive compounds (Yilmaz G, ‘Novel starch based matrices for the encapsulation and controlled release of heat sensitive compounds prepared via melt extrusion technology,” J. Controlled Release Magazine, January, 2004). Other examples include the encapsulation of volatile compounds such as essential oils, flavors, perfumes, herbicides, pesticides, pheromones, vitamins, drugs, and bacterial cells (Yilmaz G, Jongboom R O J, Feil H, and. Hennink W E, Carbohydrate Polmers, 45: 403410, 2001; Yilmaz G, Jongboom R O J, Feil H, and Hennik W E, Proc Intl Sympos Control Rel Bioact Mater., 2001; Yilmaz G, Jongboom R O J, Feil H, and Hennik, WE, “Encapsulation of living cells in starch using extrusion”, Proc Intl Sym Control Rel Bioact Mater., 2002.

### SUMMARY OF THE INVENTION

The methods and formulations of the present invention may be used to formulate superior cosmetic and personal care products containing volatile components such as fragrances, essential oils, and aroma therapy oils. The methods of the present invention may also be employed for the controlled release of oil-soluble dermatological drugs, e.g. salicylic acid, hydrophobic vitamins, e.g., vitamin A and E, plant-derived botanical extracts and other hard to formulate hydrophobic cosmetic “actives.” In all such uses, upon application to skin, the drug-loaded compositions dehydrate to occlusive starch films containing active ingredients encapsulated in oil droplets oil, which active ingredients will be slowly released by water moisture trapped by the occlusive film, moisture subsequently applied to the dried film, and/or moisture due to transepidermal water loss. The methods of the present invention may also be used to coat porous substrates, such as paper products, from which the volatile components may be released by the application of water. Methods of preparation of the present invention can be stored in air sealed containers in the lotion state with a prolonged shelf life of many years.

The present invention also includes methods of sequestering volatile oils in a thixotropic microemulsion, and in the dehydrated and/or dried films formed therefrom. Rehydration of the dried films produces an instantaneous release of the volatile components. In addition, the methods of the present invention included subsequent release of fragrance by subsequent application of water; as well as re-drying, followed by another release of fragrance, with water. The methods of the present application also include re-hydrating the dried film with water-based solution of temperature sensitive ingredients. This film may be dried to form a film with encapsulated volatile components, and temperature sensitive ingredients, which would not survive most encapsulation processes. The dried films may be milled to a fine powder, and later re-hydrated to form compositions, such as lotion compositions for topical application to the skin.

The formulations of the present invention also display thixotropic rheology changes, i.e., they form semi-solids upon standing but which become pourable gels and lotions upon moderate mechanical agitation, an important characteristic of a lotion or gel in that it minimizes dripping and provides easy application. The formulations of the present application also provide ease of spreading on skin and provide long-lasting protection against alcohol and water-based irritant chemicals.

A method of preparing topical formulations that are thixotropic emulsions and may also contain volatile fragrances are formed in a simple two-step process. Simply, a cornstarch slurry containing from 1% to 10% (W/V) of a food grade cornstarch is prepared in cold water containing a from 0.1% to 3% (W/V) of a cationic or amphoteric surfactant such as benzalkonium chloride, distearyldimonium chloride, and lecithin, respectively. If desired for moisturization, a specified amount 5% to 10% (V/V) of a humectant such as glycerol is added to the aqueous phase. The mixture is heated to between 70° C. and 80° C. with continuous stirring until all of the starch is dissolved. In certain cases, it is desirable to employ other starches such as potato and wheat starches, e.g., polysaccharides thickeners such as guar gum, agar, microcrystalline cellulose, and sodium carboxymethylcellulose, if one wishes to produce a transparent microemulsion.

The clarified starch is removed from heat, and allowed to cool to between 65° C. and 75° C., when the oil phase ingredients are blended in the aqueous phase by low shear mechanical mixing. Typical oil phase ingredients to be employed include vegetable oils (olive oil, and natural berry waxes), and synthetic oils such as mineral oil, petrolatum, squalane, and silicone oils. After a stable emulsion has formed oil-soluble volatile components can be blended in when the melt has dropped below 55° C. Control of both temperature and mixing conditions are essential for reproducible and consistent formulation. Alternatively, the volatile components may be added after the oil phase ingredients have been mixed into the clarified starch gels.

The ranges specified for the starch and polysaccharides are justified by the fact that gels formed below 1% (W/V) are watery and no gel is formed, and above 10% the gels are too viscous to be useful as lotions. The range for the oil phase ingredients is limited to 1% to 25% (V/V) as no stable microemulsion of the oil occurs above 25%. The range specified for the surfactants is consistent with a necessary and minimal amount to obtain a stable oil-in-water emulsion.

Hydrocolloid gel microemulsions containing volatile components are stable at least for three years at room temperature. The viscosity of such systems is typically a function of the starch to oil ratio starch-oil dispersions are achieved by processing at temperatures above 75° C. and are stabilized by low speed mechanical blending in the presence of low levels of a surfactant.

The method of forming stable oil-in-water thixotropic microemulsions of the present invention may be used to create a protective layer on the skin of the hands and are useful as skin barrier lotions; they form a flexible, but invisible glove-type coating, or “glove-within-a glove.” The compositions of the present invention are also alcohol resistant and moisturizing. A flexible yet, insensible film is formed by applying the moisture-activated fragrance release (MAFR) compositions of the present invention. The film so formed will tolerate multiple rinsing with 70% alcohol (ethanol), while maintaining the moisture of the underlying skin. Thus, the skin layer formed by the MAFR emulsion is a sanitary layer, which may be rinsed in alcohol. As may be easily understood, the MAFR-emulsions, when applied to the skin create a “wound dressing” skin layer, which may contain other added antimicrobial agents. In addition, a wound dressing may be formed from the dried starch film, with or without the volatile ingredients.

Among these four basic components many different natural and modified starches, many natural vegetable and synthetic oils, and cationic surfactants have been formulated. A common feature of all formulations (designated here as Thixogel) is the stability of starch-oil dispersions formed by heating and mixing starch and oil under controlled temperature and mixing conditions. Thixogel formulations are so-called because they display thixotropic viscosity changes, i.e., they form semi-solids upon standing but with become pourable gels and lotions upon moderate mechanical agitation.

This starch-based microemulsion has proved to be superior as a cosmetic vehicle, a skin protectant, and a vehicle for the delivery of hard to formulate plant actives. It also behaves as a stable lotion for delivery of novel hydrophobic actives that provides anti-irritant and anti-aging activities.

Thixotropic microemulsions are stable at least for three to five years at room temperature. The viscosity of such systems is a function of the starch to oil ratio.

Scanning electron microscope pictures (FIG. 1, BAC+) of Thixogel reveal the presence of oil droplet within a starch matrix with an average size distribution clustered around 0.5-3 microns. Previous studies have shown that the oil droplets in Thixogel system are coated with a polysaccharide shell. This shell prevents coalescence of the oil droplets and ensures emulsion stability due to two forces. The tendency of high molecular weight polysaccharide such as starches to precipitate due to their low water solubility, and the favorable systems increase in entropy and energy reduction that occurs when the starch molecules precipitate and form a carbohydrate layer on the oil droplets at the water-oil interface.

Within the range of useable starch concentrations, the concentration of oil phase ingredients also affects rheological properties. Oil concentrations below 1% produce watery emulsions with an oily skin feel. High oil concentrations above 15% to 25% (V/V) are less stable and require increased levels of emulsification with undesirable skin associated reactions.

Batch performance was certified by viscosity measurements made with a Brookfield Thermosel instrument. Thixogel formulations made with starch (4%) and Mineral Oil (8%), and Benzalkonium Chloride (1%) had a viscosity of 13, 200 (+/−100) cps at 20° C. and 2.5 rpm with a #27 SPDL blade. Under the same test condition a Thixogel formulation with 3.3% starch, 10% Petrolatum, and 0.5% Benzalkonium Chloride had a viscosity of 8,500 (+/−100) cps.

Stable gel emulsions can also be formed as above by heating the starch above 75° C. and blending in mineral oil at starch:oil ratios of 1:1 and 1:2. This can be accomplished using Pure Food Grade Powders or Waxy maize type starch. Finally, stable gel emulsions can be formed by heating the starch above 75° C. in the presence of Benzalkonium Chloride (1%) at a starch:oil ratio of 1:1 (using Pure Food Grade Powders). In special cases, the amphoteric emulsifier, lecithin can be substituted for Benzalkonium Chloride at a starch:petrolatum ratio of 1:2.

Polydimethylsiloxane fluids (viscosity range 10,000 to 60,000 cps) can be substituted for petrolatum at starch:oil ratios of 1:1 and 1:2 in the presence of Benzalkonium Chloride (1%), and in another formulation where an oxygen-carrying oil was required, perfluorodecalin, was substituted for petrolatum at a 1:2 starch:oil ratio in the presence of 1.6% Benzalkonium Chloride. All of the above formulations are generally useful as skin protectant gels. However, they may be made into moisturizing gels by simply incorporating 5% to 10% glycerol in the aqueous phase ingredients phase prior to heating and blending with the oils.

The basic starch: oil-in-water thixotropic microemulsions provide ease of spreading on skin, and long-lasting skin protection against water borne chemicals and skin irritants.

Starch is subject to both bacterial and fungal degradation. Unpreserved starch in Thixogel emulsions are mostly subject to contamination by molds. Several effective, all-purpose, natural preservatives are Tea Tree Oil and CITRICIDAL, Tea Tree Oil has recently been shown to be an effective antimicrobial agent for veterinary applications. It is readily incorporated into the oil phase ingredients of Thixogel formulations. Likewise, CITRICIDAL, an oil from grapefruit seeds, is a very effective antimycotic agent. CITRICIDAL appears to be superior to Tea Tree oil because it is less volatile and aromatic than Tea Tree Oil, and more long lasting as a preservative.

The use of low levels of Benzalkonium Chloride as an emulsifier also serves the dual purpose of inhibiting the growth of both bacteria and yeast. Thixogel starch formulations containing both Benzalkonium Chloride (0.5%) and CITRICIDAL (0.5%) have remained uncontaminated for several years.

For a full understanding of the present invention, references should be made to the following detailed description of the invention and its preferred embodiments, and accompanying figures and formulations.

### BRIEF DESCRIPTION OF THE DRAWINGS

FIG. 1. Low magnification (350×) scanning electron microscope photomicrographs comparing average oil droplet sizes for: starch-oil composite prepared by cooking with a surfactant (top, +BAC), b) starch-oil composite prepared by a jet cooking process without a surfactant (middle, −BAC), and c) a starch-oil composite prepared as above in b and stored at room temperature for greater than 6 months.

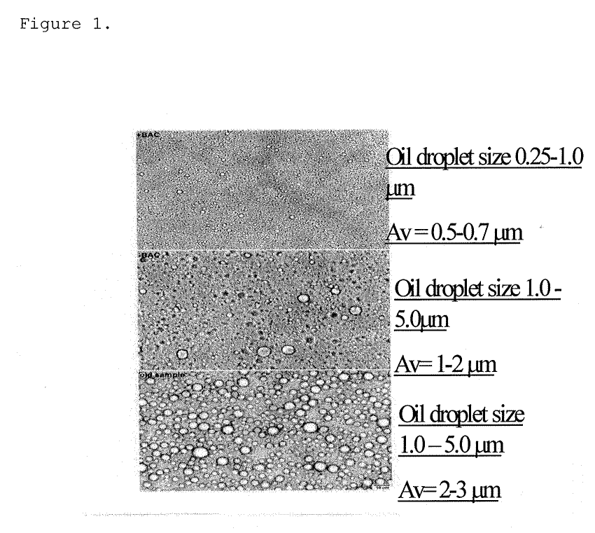

FIG. 2. Relative skin hydrating effect of three different Thixogel formulations: a) DermSeal (Formulation 1), b) EktaSeal (Formulation 2), and UltraDerm (Formulation 5).

FIG. 3. Results of crystal violet stain test for skin protection after application to right volar arm of: Vaseline (A), Thixogel formulation 1 (B), and no lotion control (C).

FIG. 4. Protective effect against acid corrosion afforded by application to aluminum foil of: A) no lotion, B) Vaseline, and C) and D), two different Thixogel formulations. Note: the white areas seen only in A and B are holes created by the corrosive effects of 3N Hydrochloric Acid after a 30-minute exposure.

FIG. 5. Photographs demonstrating the reversible effect of dehydrating and rehydrating a starch-oil thixotropic microemulsion formulation. Top) after air drying the dehydrated gel to a thin film. Bottom) after rehydrating the dehydrated gel.

FIG. 6. The kinetics of oxygen release. Data are shown for: water (⋄), water plus 10% PFC (▪), starch (4%, [symbol] ), starch (4%)+PFC (▴) and OxyTega ().

FIG. 7. Photograph showing a sample wound dressing prepared from a dehydrated starch-oil composite film affixed to sterile gauze.

### DETAILED DESCRIPTION OF THE INVENTION

### Example 1

### Emulsification Studies on Thixogel Formulations

The effect of cationic surfactants in stabilizing starch emulsions was studied. Benzalkonium Chloride above 0.13% are effective by themselves. In other studies, pairs of emulsifiers have been substituted. One pair consisted of 0.5% Oleic Acid combined with 0.1% Benzalkonium Chloride. Another pair examined was 0.5% Palmitoleic Acid and 0.1% Benzalkonium Chloride. These pairs require special processing as ion-pairs can be formed between anion and cationic members of the pair when heated during the pre-gelatinization step. This was avoided by altering the pH as indicated above. To a limited extent, addition of 0.5% CITRICIDAL can also lower the concentration of required emulsifiers.

Table 1 presents a summary of results evaluating the ability of various surfactant and fatty acids and oil to form stable Thixogel type emulsions. The test results indicate that Benzalkonium Chloride (BC) combined with oleic acid at 0.5% or higher and the combination of BC and starch (20%) is capable of forming a stable emulsion of soybean oil and water, even at 0.1% Benzalkonium Chloride. Lastly, the combination of Oleic Acid (1%) and CITRICIDAL (0.5%) was found to be effective in producing stable oil-in-water emulsion. By itself, Oleic Acid was found to be ineffective at stabilizing the emulsions.

In another series of investigations, the ability of several formulations to act as emulsifiers, themselves, was examined. In this assay, 0.2 ml of each formulation was added directly to a test tube containing 2.0 ml of Soy Bean Oil carefully layered on top of 2.0 ml of water. The mixture was then shaken to thoroughly mix the two phases. A control containing just the two phases was used as a reference for measuring the extent of emulsification and the resulting stability of the emulsions.

Addition of 0.2 ml of a Thixogel formulation containing Starch and Mineral Oil in a 1:2 ratio, in the presence of Benzalkonium Chloride, led to the appearance of an interface after 60 minutes. A transfer of 20% of the water phase into the oil phase was accomplished with this approach. In a second formulation, Starch and Petrolatum (in a 1:2 starch:oil ratio) in the presence of 0.1% Benzalkonium Chloride, and 0.5% Palmitoleic Acid generated only a 10% shift if water into the oil phase. Finally, a similar formulation, containing 4% Dimethicone, produced a 5% transport of oil into the water phase. These results suggest that emulsification can be brought about by both movement of oil into the water phase and by movement of water into the oil phase of such systems.

TABLE 1
The role of different model components of the
starch-oil composite on the emulsification of
soy bean oil in a two phase oil:water mixture.

| Benzalkonium | | | |
|---|---|---|---|
| Chloride | Oleic Acid | Citricidal | Starch |
| (Wt. %) | (Wt. %) | (Wt. %) | (20%) |
| 1.0 | 0 | 0 | none |
| 1.0 | 0 | 0 | added |
| 0.5 | 1.0 | 0.5 | none |
| 0.5 | 0.5 | 5.0 | None |
| 0.1 | 0.5 | 0 | added |
| 0.1 | 0 | 0 | added |
| 0 | 1.0 | 0.5 | none |

### Example 2

### Skin Hydrating Formulations

The following five formulations (DermSeal—# F1, Aqua Seal—# F2, VegaSeal—# F3, EktaSeal—# F4 and EktaDerm—#F5) are basic skin barrier gels and lotions that possess good skin protecting and skin moisturizing properties. These model formulations have been tested by a variety of tests including skin hydration using a device that measures skin capacitance, the Corneometer (Model CM 825, Courage & Khazaka, Koln, Germany. Formulation 14 employs squalane as the only oil phase ingredient. It has been cited as an emollient oil with low irritancy potential and has some skin hydrating action by itself.

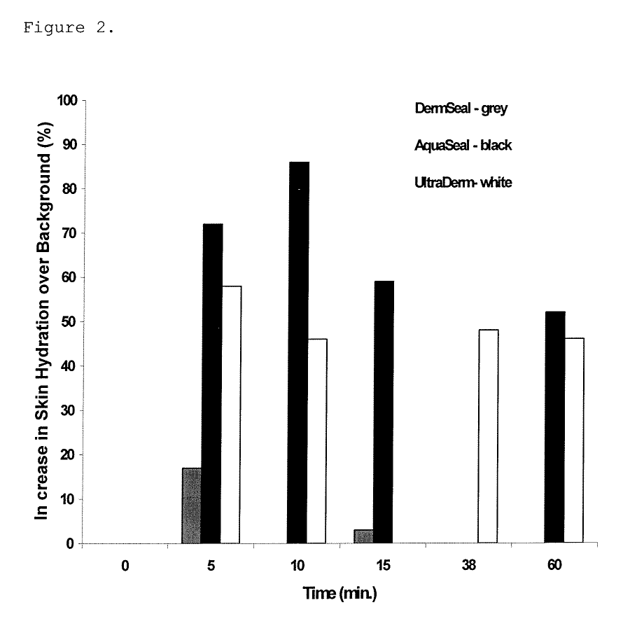

FIG. 2 shows that formulation F1 has virtually no effect on skin hydration, while formulation F2 significantly elevates skin moisture to levels 50% greater than that seen in normally hydrated skin. The elevated skin moisture obtained persisted for at least one hour after application of this formulation at 26° C. and a relative humidity of 28%. Similarly, formulation F5 with 10% Glycerol provides significant elevation of skin hydration.

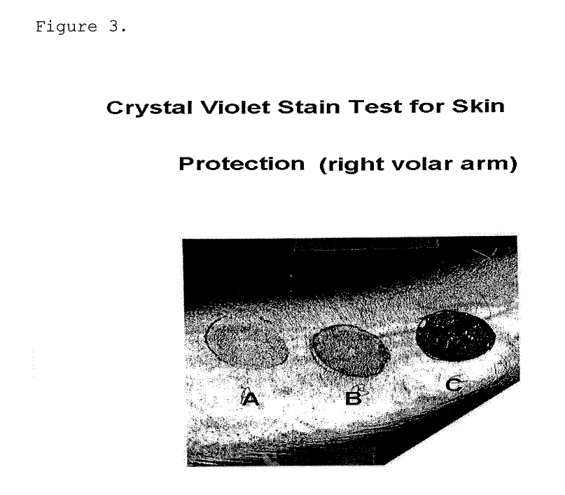

The skin-protecting effect of formulation F1 was demonstrated by the crystal violet stain test as describe here. Several 2.5 cm² circles are drawn on the volar arm surface of a human subject. The encircled areas are then coated with test material (A, Vaseline; B, formulation F1 (Tx−1D), or C) no coating material (unprotected control). Discs of filter paper are then dipped into a 0.2% crystal violet stain solution, drained of excess dye, and applied to the treated areas for 5 minutes. The discs were then removed and the excess dye washed off by several water rinses. The resulting stained skin areas were then photographed. A typical result is shown in FIG. 3 below. Clearly, both Vaseline and formulation F1 (DermSeal) were effective.

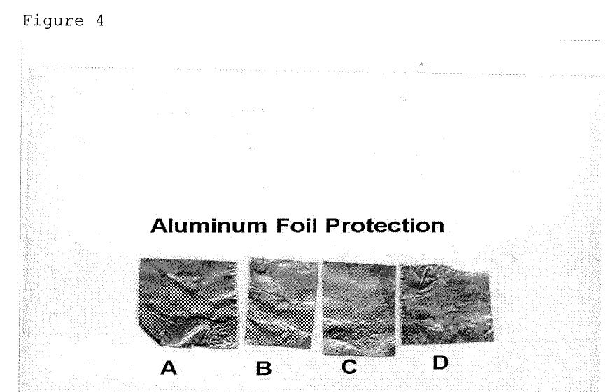

The skin-protecting effect of formulation F1 was also demonstrated by conducting a modification of the aluminum foil deterioration test. In this assay, pieces of aluminum foil are first coated with 50 microliters of the test gel and air dried for 10 minutes. The coated foil area is then exposed to 100 microliters of 3N HCl acid for 30 minutes. The results of one such test is presented in FIG. 4. The control (A) piece of foil developed a small hole. By contrast, a variant of formulation F1 (C), composed of 4% starch, 8% Mineral Oil and 1% Benzalkonium Chloride, and formulation F5, an emulsion composed of 4% natural starch, 8% Petrolatum, 5% Mineral Oil, 1% Polysiloxane, 4% Dimethyl cyclopentasiloxane, 10% glycerol, and 0.5% Benzalkonium Chloride, did not develop any holes. By contrast, Petrolatum alone when applied to aluminum foil did not prevent the development of holes (B).

### Example 3

### Reversible Hydration Effects of Topically Applied Thixogel Formulations

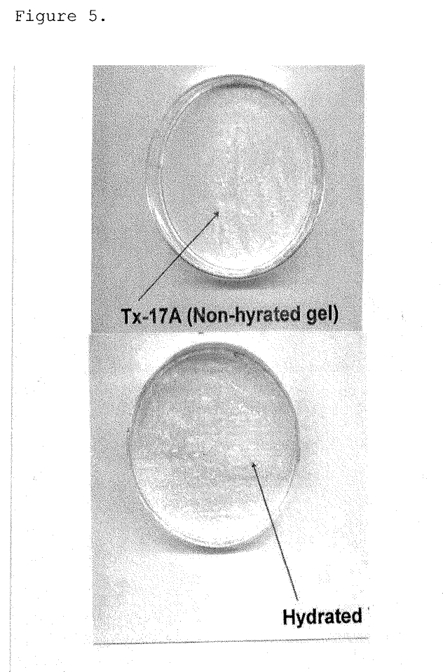

A remarkable property of all Thixogel formulations is their ability to be air-dried and then to rehydrate back to their original volume, upon addition of water. This is seen for a sample of formulation F5 as shown in FIG. 5 below.

This phenomenon occurs when the gel is applied to skin. After drying, it can be rehydrated with water, and this can be repeated through many cycles of drying and rehydration. Moreover, upon drying on the hands, they may be rinsed in 70% ethanol and air-dried without preventing rehydration upon subsequent exposure to water. This unique property we have called, a “glove in a glove.” It may have wide ranging benefits for healthcare workers who get dry irritated skin because they repeatedly wash their hand multiple times a day often employing intervening alcohol washes.

### Example 4

### Delivery of Oxygen from a Thixogel Formulation

It was speculated that starch-coated oil droplets might bind and then slowly release dissolved oxygen. Oxygen may be incorporated in such systems by using Perfluorodecalin as an oil. This material is widely used to bind oxygen and as a blood substitute. It has been incorporated into emulsions in a number of patents which are herein mentioned are: Moore. U.S. Pat. No. 4,569,784, 1986; Gianladis. U.S. Pat. No. 3,277,013, 1966; Rosano et al. U.S. Pat. No. 3,778,381 (1973, Samejima et al. U.S. Pat. No. 3,823,091,1974, Yokoyoma et al. U.S. Pat. No. 3,993,581, 1976; White, U.S. Pat. No. 4,366,169, 1982; and Arnaud and M. Mellul. FR No. 2688006A1, 1993. They disclose the use a perfluorocarbon to bind oxygen and to deliver it in a formulation.

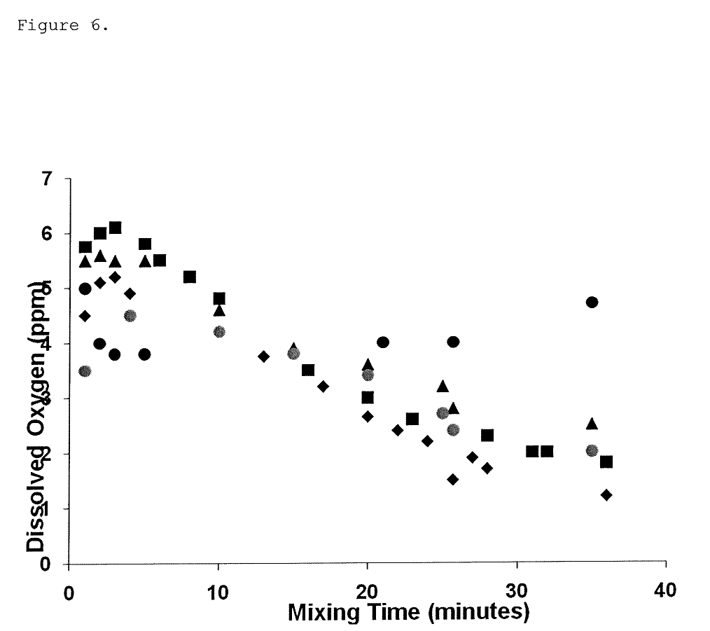

Here, we dissolved oxygen into a Thixogel formulation F6 by replacing all other oil phase ingredients with 10% Perfluorodecalin (PFC). This formulation is called OxyTega. In order to achieve this effect, various aqueous solutions were oxygen charged. These were composed of just one added component or OxyTega gel, itself. Oxygen was bubbled directly into the solution for 5 minutes at 20 psi in an open-air container. The oxygenated solutions obtained were then continuously stirred at 25° C., at moderate speed and dissolved oxygen was continuously monitored with an oxygen electrode connected to an oxygen meter. The results are summarized in FIG. 6.

The kinetic curves for all Thixogel components, with or without Perfluorodecalin share a similar oxygen release rate and have an approx. half-life of 15 minutes. By contrast, OxyTega based systems retain the dissolved oxygen over the 30 minutes. There is, in fact, a trend toward increasing the amount of oxygen available for release beyond 30 minutes. Similar tests conducted on Thixogel emulsions employing Mineral oil and 1% Benzalkonium chloride shows a half-life of approximately 90 minutes. The most favorable starch/mineral oil ratio for achieving slow oxygen release occurred at ratio of 1:3.

### Example 5

### Anti-Microbial Thixogel Formulations

Benzalkonium Chloride, at 0.5%, acts as both a surfactant and anti-bacterial in Thixogel formulations. Given concerns about possible skin irritation at or above 0.5%, the concentration was reduced to 0.1%. Palmitoleic Acid was also added to supplement the emulsifying action and, at the same time, to increase the overall anti-microbial action of the Benzalkonium Chloride/Palmitoleic Acid combination.

SanoSeal Gel, formulation F8 was tested for its bactericidal action on a clinical isolate of Staphylococcus aureus. The bacteria were applied at a level of 5×10⁵ cells to a saline moistened sterile filter paper and exposed for 20 minutes to formulation F8 (100 μL per filter) to completely cover the bacterized paper. Controls included sterile filter papers with an equal number of bacteria. These were covered with a sterile starch/oil dispersion lacking Palmitoleic acid (positive control). Sterile filter papers, with no bacteria and covered with sterile formulation F8 were also employed as controls. After treatment, the filter papers were aseptically transferred to a sterile broth and incubated on a rotary shaker overnight. It was found that bacterized paper without Palmitoleic acid in Thixogel was clouded by growth of bacteria. By contrast, filter papers either without bacteria or coated with SanoGel were as clear as uninoculated sterile broth. Small aliquots from each broth were then transferred to a fresh sterile broth and incubated again overnight at 37° C. Only the cloudy broth from the bacterized Thixogel-treated flask grew out bacteria. These results show that formulation F8 (SanoSeal Gel) kills up to five-logs of applied bacteria in a 20-minute exposure. Since the formulation contains no toxic chemicals, and no drying alcohol, it is effective and safe and is also not harsh or irritating to skin.

### Example 6

### Delivery of Anti-Oxidant Botanicals

Formulations F9 through F12 were chosen as the best delivery systems for a hydrophobic plant active for the following reasons: 1) hydrophobic plant active compounds are soluble in oil phase ingredients, 2) dry powders can be prepared by exhaustive venting of volatile solvents, 3) dry powders of hydrophobic plant active compounds are soluble in mineral oil, and plant actives dissolved in mineral oil are also soluble in combined oil phase ingredients of formulation 9. In addition, protection of plant anti-oxidants from light and air can be achieved by adding Tocopherol (Vitamin E) directly to the mineral oil prior to dissolving the plant active. We have discovered several good anti-oxidant plant extracts as candidates for incorporation into our chosen Thixogel hydrophobic delivery system (Formulation F5, EktaDerm). Moreover, it is our claim that plant extracts with strong antioxidant activity will be useful sources of plant-derived anti-irritants.

The anti-oxidant activity of anti-irritant plant extracts was assayed by the diphenylpicrylhydrazyl radical (DPPH*) test (Bonina et al, 2002). Table 2 summarizes these results.

TABLE 2
Relative free radical scavenging activity of botanical extracts
incorporated in formulation F9 as determined by a modification
of previously published methods (Bonina et al, 2002).

| | Antioxidant | Activity relative |
|---|---|---|
| Botanical Extract | Activity* | to Vitamin E** |
| Indole acetic acid | 2.0 | 4.1 |
| Green onion leaf | 3.1 | 6.7 |
| Red Swiss chard | 4.0 | 5.2 |
| Tomato paste | 13.3 | 1.6 |
| Corn tassel | 16.5 | 1.3 |
| Autumn olive berry | 176 | 0.1 |

(Values given are from diphenylpicrylhydrazine (DPPH) assay measurements).
*Antioxidant potency = EC₅₀ × concentration(kg/liter of plant extract.
**Vitamin E units, 1 unit = EC 50 of 46 μmoles

In our search for a good plant-derived anti-oxidant, Autumn Olive (Elaeagnus umbellata) was found to be a very rich source of anti-oxidants, as were Cranberry juice, and grapefruit seed oil (CITRICIDAL). Two other sources of anti-oxidants were found to be hydroalcoholic extracts of corn tassels (Tasselin) and tomato paste. In addition, we have also isolated lycopenes from both tomato paste and Autumn Olive berries. They are both rich sources of carotenes, and have been incorporated into Formulations 10 and 11 (PhytoSeal Gels). Similarly, hydroalcoholic extracts of green onion leaves and red Swiss chard have demonstrated modest but significant anti-oxidant activity. Green onion leaf extract was incorporated into Formulation 12 along with Retinyl Acetate to enhance the anti-oxidant properties of this formulation.

### Example 7

### Moisture Activated Fragrance Release

It can be understood that there are many volatile fragrances available for moisture activated fragrance release from various starch-oil thixotropic gel formulations. By way of example we have chosen two fragrances, phenethyl alcohol (PEA) and Lavender oil (LO). Typically, 0.5% PEA was incorporated into the oil phase ingredients of formulations 13, and 15-20 (see Example 14, List of Formulations). Alternatively, 0.5% PEA was introduced to the hot melt of the starch-oil microemulsions immediately after blending of the oil phase ingredients. Likewise, LO was either added directly into the oil phase ingredients prior to mixing with the aqueous phase ingredients or immediately after blending of the aqueous phase ingredient with the oil phase ingredients.

### Fragrance Test:

Bibulous paper was cut into 1″ squares and impregnated with approximately 2 mg/ml of the fragrance-loaded Thixogel lotions. The impregnated papers were air-dried and stored in airtight sealed wrappers at room temperature under dehumidified conditions for varying lengths of time. To initiate fragrance release from the air-dried specimens, the impregnated papers were placed on dry paper toweling and small aliquots of water allowed to infiltrate the bibulous paper.

### Results:

A panel of six blinded subjects were asked to smell a series of wetted bibulous papers 5 minutes after the papers were wetted. As a control, bibulous papers were impregnated with vehicle lotions that did not have any added fragrances. Table 3 present the results of this test panel.

TABLE 3
Sensory Assessment of PEA-Impregnated
Fragrance Release by Panel Subjects

| Subject No. | Control papers | PEA papers |
|---|---|---|
| 1 | No | Yes |
| 2 | No | Yes |
| 3 | No | Yes |
| 4 | No | Yes |
| 5 | No | Yes |
| Total 6 | All no | All yes |

Yes, detected the rose-like smell; No, did not detect rose-like smell

In a second test, bibulous paper was impregnated with Lavender Oil-containing Thixogel lotions and air-dried papers stored for 45 days. Again, a panel of six blinded subjects was asked to determine which of the wetted papers, control or PEA-impregnated samples gave off a distinct rose-like fragrance. The results were identical to those disclosed in Table 3. All of the subjects correctly identified the rose-like fragrance only from the PEA-impregnated papers. Similar results were obtained from human panel studies using Lavender oil containing starch-oil microemulsion lotion impregnated papers versus un-impregnated controls. As a further control, bibulous papers were impregnated with 0.5% PEA or in oleophilic base containing Lavender oil. When these papers were stored for 7 days or greater, no PEA-like or Lavender oil-like scent could be detected by a panel of six blinded subjects.

### Example 8

### Repeated Cycles of Fragrance Release from Once Impregnated Papers

In another study, bibulous papers were impregnated with PEA-containing thixotropic microemulsion lotions allowed to air dry and stored under dehumidified conditions at room temperature for 7 days. On day 8, the papers were wetted with water and were found to release fragrance as predicted from the above results. The wetted papers were air dried again and stored for an additional 7 days. When rewetted with water these once-wetted papers again gave off a distinct rose-like fragrance indicative of moisture-activated fragrance release. This, too, was confirmed by a panel of six-blinded subjects. In fact, fragrance release can be elicited repeatedly from the same piece of impregnated paper through several cycles of air-drying and moisture exposure.

### Example 9

### Release of Fragrance from Skin

A thixotropic microemulsion lotion (formulation F13) was loaded with 0.5% PEA an applied to the volar arm skin of several subjects. The lotion was allowed to dry on the skin for 30 minutes until no further scent could be detected. In all three subjects, scent could be restored by spraying a fine mist of water on the fragrance-treated skin areas.

### Example 10

### Effect of Other Releasing Agents Beside Water

Bibulous papers were impregnated with PEA-containing thixotropic microemulsion lotion (Formulation 1) air dried and stored for 14 days. The fragrance test was conducted using different moisturizers as described in Table 4 below.

TABLE 4
Effect of Different Moisturizing Solvents on Fragrance Release.

| Solutions tested | Fragrance released |
|---|---|
| Water | Yes |
| 10% PG | Yes |
| 14% IP | Yes |
| 70% ETOH | No |

Solutions Prepared: 1)10% (v/v) of Propylene glycol(PG) in water, 2) 14% (v/v) isopropyl alcohol (IP) in water, 3) 70% (v/v) of ethanol in water (ETOH), and 4) pure deionized water.

Fragrance was detected within 5 minutes after duplicate pieces of bibulous papers were wetted with the different solvents except IP which has its own scent. Once that had blown off the rose-like fragrance could be detected. Thus, typical water-based solvents may be used to accomplish the water release of the volatile components from the dried (or dehydrated) films.

### Example 11

### Fragrance Release from Dehydrated Gels and Powders

### Preparation of Dehydrated and Rehydrated Gels:

Formulation F13 was prepared and one volume of it was diluted with two volume of deionized water. The diluted lotion/gel mixture was thoroughly mixed by stirring slowly under mild heating (60° C.) until a homogenously mixture was obtained. The diluted lotion containing about 1% starch and 3.3% oil; it was cast to a depth of 0.5 mm into a clean Petri dish and allowed to solidify into a solid gel overnight (about 20 hours) at room temperature. The solidified gels so formed are firm and non-pourable. In order to form an elastic gel the solidified cast gels were dehydrated by layering a sufficient amount of 50% Ethanol on top of the gelated surface for 19 hours. The gels undergo about 10% shrinkage in total surface area (see FIG. 2), and as a result of dehydration they are pliable and can be easily removed from the Petri dish with sterile forceps. When such dehydrated gels are removed to another glass Petri dish they can be further air-dried to a thin dry film that has less than 10% of the original weight of the original hydrated cast gel. Such dry and ethanol dehydrated gels will almost instantly rehydrate to their original weight when placed in a sufficient amount of distilled water. This reversible hydration-dehydration process occurs without any appreciable loss of starch or oil. Further, it is possible to study fragrance release from dehydrated gels if one first loads the original formulation F5 with a water-insoluble fragrance, such as PEA.

When, a dehydrated gel was prepared with formulation 13 (ThixoDerm-F) and assessed for its content of PEA, it was found that such dehydrated gels retain greater than 80% of the total concentration of PEA fragrance.

These results indicate that fragrance can be first encapsulated in the oil droplets in a starch matrix and that remains encapsulated even after alcohol dehydration and drying to a thin film. The technique of forming dry film with entrapped fragrances is a useful property for coating of artificial substrates, e.g., glossy paper for printing, from which a delayed release of fragrance is esteemed desirable. In addition, the dry films can be further processed to a powder by milling of the flaked films to a fine powder. Large scale processing of powders can be further accomplished by drum drying, flaking and milling with starch-oil composites that contain as much as 30% oils. “Instant” lotions may be formulated by rehydrating these powders to a lotion consistency.

Fragrance Release from Dehydrated Gels:

Thin dry films of ThixoDerm-F were prepared as described above and were sprayed with a fine mist of water. A bloom of fragrance was readily detected within a few minutes of exposure to the water moisture.

### Uptake of Water-Soluble Botanical Extracts at Ambient Temperatures:

Another application of the invention is the rehydration of dry films with aqueous solutions containing an active agent that is thermo-sensitive. The dry film will take up aqueous solutions of many water-soluble topical actives such as water-soluble botanical extracts that may lose much of their activity during heat processing steps required in the preparation of cosmetic emulsions. The dried films can take up water-soluble antioxidants e.g., ascorbic acid (Vitamin C) without exposure to harsh conditions associated with heat processing, or emulsification with strong surfactants. The films may be subsequently dried, milled to a powder, and used to make “instant” lotions. Alternatively, the dried milled films described in 2 above may be rehydrated with a water-based solution containing the temperature sensitive ingredients, to form a composition, or an “instant” lotion, containing both encapsulated oil with volatile components and temperature sensitive ingredients which to not survive most encapsulation processes.

### Example 12

### Release of a Volatile Insect Repellent from Skin

The methods of forming a thixotropic microemulsion outlined for the preparation and release of fragrances was modified for the sequestering and release of the insect repellent DEET (N, N-diethyl-m-toluamide). In this formulation the entire oil phase to form the microemulsion is from 3% to 25% (V/V) neat DEET. The lotion so formed can be applied directly to the skin of an animal as a spray or as a lotion to the skin of a human.

### Example 13

### Preparation of a Thixogel Wound Dressing

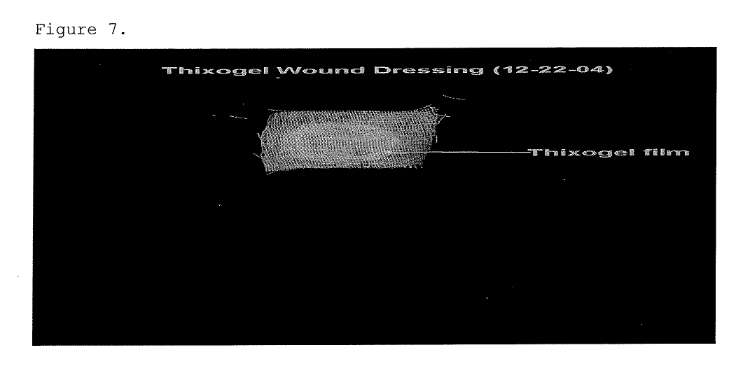

The dehydrated or dried films of the present invention, such as that shown in FIG. 2 may find use as wound dressings. The dried film can absorb up to 8× its weight in water, and can be used to absorb wound exudates. If desired the films may have typical wound healing ingredients incorporated therein, such as anti-microbials, or warming components such as camphor or for pain relief such as capsaicin. As the films are not skin adherent, it may be desirable to provide a backing, such as a nonwoven or woven fabric backing, which may be secured, as with an adhesive tape; or a film-type backing with an adhesive layer. Alternatively, the compositions of the present invention may be coated on the fabric backing before dehydrating or drying, to form a fibrous coating rather than a film. FIG. 7 illustrated wound dressing formed from a dried film made according to the present invention, and a gauze (fabric) backing.

### Example 14

### List of Formulations and Preparation Procedures

The method of preparation is given below.

### F1. DermSeal, a Basic Skin Barrier Gel

| | |
|---|---|
| Ingredient | Wt. % |

| | | |
|---|---|---|
| A. | Petrolatum jelly | 7.5 |
| B. | Deionized Water | 88.0 |
| | Corn Starch | 3.5 |
| | Benzalkonium Chloride | 0.5 |
| C. | Citricidal | 0.5 |

Weigh the Part A ingredient and heat at 50° C. until thoroughly melted in a suitable vessel equipped with a mixer. Add C ingredient to pre-heated Part A ingredient. Weigh the Part B starch ingredient, and place in a suitable vessel equipped with low-shear mixer. Add a sufficient volume of deionized water to produce a 0.5% concentration of benzalkonium chloride. Heat the Part B ingredients at 80° C. until the starch is entirely dissolved. Remove from heat, add directly to heated Part A ingredient and then heat at 65° C. with continuous mixing until a homogeneous emulsion is formed.

### F2. EktaSeal, a Skin Barrier and Moisturizing Gel

| | |
|---|---|
| Ingredient | Wt. % |

| | | |
|---|---|---|
| A. | Petrolatum jelly#@ | 8.8 |
| B. | Deionized Water | 77.0 |
| | Corn Starch | 3.6 |
| | Benzalkonium Chloride | 0.1 |
| | Glycerol | 10.0 |
| C. | Citricidal | 0.5 |

Add a sufficient volume of deionized water, glycerol, and benzalkonium chloride, and heat the Part B ingredients at 80° C. until the starch is entirely dissolved. Remove from heat and add to pre-heated Part A and Part C ingredients. Heat at 65° C. with continuous mixing until a homogeneous emulsion is formed.

### F3. VegaSeal, an all Natural Skin Moisturizing Gel

| | |
|---|---|
| Ingredient | Wt. % |

| | | |
|---|---|---|
| A. | Soy Bean Oil | 8.5 |
| | Lecithin | 1.0 |
| | (dissolved in 10% ethanol) | |
| B. | Deionized Water | 77.0 |
| | Corn Starch | 3.0 |
| | Glycerol | 10.0 |
| C. | Citricidal | 0.5 |

Heat Part B aqueous phase ingredients at 80° C. until the starch is entirely dissolved. Mix the combined Part B ingredients with Part A and Part C ingredients. Heat the combined oil-water mixture at 65° C. with continuous mixing until a homogeneous emulsion is formed. Meadowfoam Oil, Oleic Acid, Olive Oil and Canola Oil can be substituted for Soy Bean Oil.

### F4. SilkDerm, a Moisturizing Skin Barrier Lotion

| | |
|---|---|
| Ingredient | Wt. % |

| | | |
|---|---|---|
| A. | Dimethicone (200 ® Fluid) | 5.5 |
| B. | Deionized Water | 80.5 |
| | Corn Starch | 3.4 |
| | Benzalkonium Chloride | 0.1 |
| | Glycerol | 10.0 |
| C. | Citricidal | 0.5 |

Add sufficient volume of deionized water, glycerol and benzalkonium chloride, mix thoroughly, and heat the Part B ingredients at 80° C. until the starch is entirely dissolved. Remove from heat and add Citricidal to Part A ingredient. Heat at 65° C. with continuous mixing until a homogeneous emulsion is formed.

### F5. EktaDerm, a Basic Topical Delivery System

| | |
|---|---|
| Ingredient | Wt. % |

| | | |
|---|---|---|
| A. | Poly(dimethylsiloxane) | 0.8 |
| | (DC-200 Fluid ®) | |
| | Decamethyl Cyclopentasiloxanes | 3.2 |
| | (DC-245 Fluid ®) | |
| | Mineral Oil | 4.0 |
| | Petrolatum Jelly | 9.3 |
| B. | Deionized Water | 69.0 |
| | Corn Starch | 3.1 |
| | Benzalkonium Chloride | 0.1 |
| | Glycerol | 10.0 |
| C. | Citricidal | 0.5 |

Add a sufficient volume of deionized water, glycerol and benzalkonium chloride, mix thoroughly, and heat the Part B ingredients at 80° C. until the starch is entirely dissolved. Remove from heat and cool to 65° C. Weight out Part A ingredient (mineral oil, DC-200, DC-245 and petrolatum jelly) and add directly to pre-heated Part B ingredients. Stir in Part C ingredient, and mix continuously until a homogeneous emulsion is formed.

### F6. OxyTega, an Topical Oxygen Delivery Gel

| | |
|---|---|
| Ingredient | Wt. % |

| | | |
|---|---|---|
| A. | Perflurodecalin | 12.5 |
| B. | Deionized Water | 82.5 |
| | Corn Starch | 4.0 |
| | Benzalkonium Chloride | 0.5 |
| C. | Citricidal | 0.5 |

Add a sufficient volume of deionized water, and add the benzalkonium chloride. Mix thoroughly, and heat the Part B ingredients at 80° C. until the starch is entirely dissolved. Remove from heat and add Part C ingredient to Part A ingredient (Perfluorodecalin) and heat at 65° C. with continuous mixing until a homogeneous emulsion is formed. Perfluorodecalin (95%, Aldrich Company, Milwaukee, Wis. 53201).

### F7. Itch-Relief Gel and Witch Hazel Delivery System

| | |
|---|---|
| Ingredient | Wt. % |

| | | |
|---|---|---|
| A. | Petrolatum Jelly | 8.0 |
| | Poly(dimethylsiloxanes) | 1.0 |
| B. | Potato Starch | 4.0 |
| | Benzalkonium Chloride | 1.0 |
| | Hammelis Water(86% witch hazel) | 71.5 |
| | (86%, Witch Hazel) | |
| | Isopropyl Alcohol | 14.0 |
| C. | Citricidal | 0.5 |

Add a sufficient volume of Hammelis Water (14% isopropyl alcohol), and benzalkonium chloride. Mix thoroughly and heat the Part B ingredients at 80° C. until the starch is entirely dissolved. Remove from heat and add Part C ingredient to Part A ingredient (Petrolatum jell) and heat at 65° C. with continuous mixing until a homogeneous emulsion is formed.

### F8. SanoSeal Gel, an Antimicrobial Hand Lotion

| | |
|---|---|
| Ingredient | Wt. % |

| | | |
|---|---|---|
| A. | Petrolatum jelly | 6.6 |
| | Poly(dimethylsiloxanes) | 5.0 |
| | Palmitoleic Acid | 0.5 |
| B. | Deionized Water | 84.0 |
| | Corn Starch | 3.3 |
| | Benzalkonium Chloride | 0.1 |
| C. | Citricidal | 0.5 |

Add a sufficient volume of deionized water and benzalkonium chloride, mix thoroughly, and heat the Part B ingredients at 80° C. until the starch is entirely dissolved. Remove from heat and add Part C ingredient to Part A ingredient (Petrolatum Jelly, Dimethicone, and Palmitoleic Acid), and heat at 65° C. with continuous mixing until a homogeneous emulsion is formed.

F9. PhytoSeal L, an anti-irritant Botanical Topical Delivery System

| | |
|---|---|
| Ingredient | Wt. % |

| | | |
|---|---|---|
| A | Petrolatum Jelly | 9.0 |
| | Poly(dimethylsiloxane) | 0.8 |
| | Decamethyl cyclosiloxane | 3.2 |
| | Mineral Oil | 4.0 |
| B | Deionized Water | 69.0 |
| | Corn Starch | 3.1 |
| | Benzalkonium Chloride | 0.2 |
| | Glycerol | 10.0 |
| C | Tomatopaste extract | 0.1 |
| D | Citricidal | 0.5 |
| | Tocopherol | 0.1 |

Add a sufficient volume of deionized water, glycerol and benzalkonium chloride, mix thoroughly, and heat the Part B ingredients at 80° C. until the starch is entirely dissolved. Remove from heat and add Part D ingredient (Tocopherol and CITRICIDAL) to Part A ingredient (Petrolatum Jelly, 200® Fluid, 245® Fluid). Part C ingredient (lycopene solution in mineral oil) is added to Part A ingredients and heated at 65° C. and then added to Part B ingredients with continuous mixing until a homogeneous emulsion is formed.

### F10. PhytoSeal C/L, a Photo-Aging Skin Repair Gel

| | |
|---|---|
| Ingredient | Wt. % |

| | | |
|---|---|---|
| A. | Petrolatum Jelly | 9.2 |
| | Poly(dimethylsiloxane) | 0.8 |
| | Decamethyl cyclosiloxane | 3.2 |
| | Sea Buckthorn Oil | 4.0 |
| B. | Deionized Water | 68.0 |
| | Corn Starch | 3.2 |
| | Benzalkonium Chloride | 0.8 |
| | Glycerol | 10.0 |
| C. | Carrot Extract | 0.1 |
| | TomatoPaste Extract | 0.1 |
| D. | Citricidal | 0.5 |
| E. | Tocopherol | 0.1 |

Add a sufficient volume of deionized water, glycerol and benzalkonium chloride, mix thoroughly, and heat the Part B ingredients at 90° C. until the starch is entirely dissolved. Remove from heat. Add Part D and E ingredients (Tocopherol and Citricidal) to Part A ingredients (Petrolatum Jelly, 200® Fluid, 245® Fluid, Mineral Oil) at 65° C. and mix Part B ingredients with continuous stirring until a homogeneous emulsion is formed.

### F11. PhytoSeal T, an Anti-Aging Botanical Topical Delivery System

| | |
|---|---|
| Ingredient | Wt. % |

| | | |
|---|---|---|
| A. | Petrolatum Jelly | 9.2 |
| | Poly(dimethylsiloxane) | 0.8 |
| | Decamethyl cyclosiloxane | 3.2 |
| | Mineral Oil | 4.0 |
| B. | Deionized Water | 68.0 |
| | Corn Starch | 3.2 |
| | Benzalkonium Chloride | 0.8 |
| | Glycerol | 10.0 |
| C. | Corn Tassel Extract | 0.1 |
| | Retinol | 0.1 |
| D. | Citricidal | 0.5 |
| E. | Tocopherol | 0.1 |

Add a sufficient volume of deionized water, glycerol and benzalkonium chloride, mix thoroughly, and heat the Part B ingredients at 80° C. until the starch is entirely dissolved. Remove from heat. Add Part D ingredient (Tocopherol and Citricidal) to Part A ingredients (Petrolatum Jelly, 200® Fluid, 245® Fluid, Mineral Oil) at 65° C. and mix Part B ingredients with continuous stirring until a homogeneous emulsion is formed.

### F12. PhytoSeal R/O, an Anti-Wrinkling Botanical Topical Delivery System

| | |
|---|---|
| Ingredient | Wt. % |

| | | |
|---|---|---|
| A. | Petrolatum Jelly | 9.2 |
| | Poly(dimethylsiloxanes) | 0.8 |
| | Decamethyl cyclosiloxanes | 3.2 |
| | Mineral Oil | 4.0 |
| B. | Deionized Water | 68.0 |
| | Corn Starch | 3.2 |
| | Benzalkonium Chloride | 0.8 |
| | Glycerol | 10.0 |
| C. | Retinyl Acetate | 0.1 |
| | Onion Leaf Extract | 0.1 |
| D. | Citricidal | 0.5 |
| E. | Tocopherol | 0.1 |

Add a sufficient volume of deionized water, glycerol and benzalkonium chloride, mix thoroughly, and heat the Part B ingredients at 90° C. until the starch is entirely dissolved. Remove from heat. Add Part D and E ingredients (Tocopherol and Citricidal) to Part A ingredients (Petrolatum Jelly, 200® Fluid, 245® Fluid, Mineral Oil) at 65° C. and mix Part B ingredients with continuous stirring until a homogeneous emulsion is formed.

### F13. Thixoderm-F, a Natural Emollient Fragrance Release Delivery System

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Mineral Oil | 4.1 |
| | Berry Wax | 9.3 |
| B. | Deionized Water | 72.9 |
| | Corn Starch | 3.2 |
| | Benzalkonium Chloride | 0.5 |
| | Glycerol | 5.0 |
| C. | Citricidal | 0.5 |
| D. | Phenethyl Alcohol | 0.5 |

Add a sufficient volume of deionized water, glycerol and benzalkonium chloride, mix thoroughly, and heat the Part B ingredients at 90° C. until the starch is entirely dissolved. Remove from heat and cool to 65° C. Weight out Part A ingredient (mineral oil, DC-200, DC-245 and Berry Wax/Olive Oil, EnviroPure310, React-NTI) and add directly to pre-heated Part B ingredients. Stir in Part C and D ingredient, and mix continuously until a homogeneous emulsion is formed.

### F14. OLIVADERM, a Natural Emollient Topical Delivery System

| | |
|---|---|
| Ingredient | Wt. % |

| | | |
|---|---|---|
| A. | Squalane | 7.5 |
| B. | Deionized Water | 83.1 |
| | Corn Starch | 3.3 |
| | Benzalkonium Chloride) | 0.1 |
| | Glycerol | 5.0 |
| C. | Citricidal | 0.5 |
| D. | Phenethyl Alcohol | 0.5 |

Add a sufficient volume of deionized water, glycerol and benzalkonium chloride, mix thoroughly, and heat the Part B ingredients at 80° C. until the starch is entirely dissolved. Remove from heat and cool to 65° C. Weight out Part A ingredient (Squalane, Vegetal) and add directly to pre-heated Part B ingredients. Stir in Part C and D ingredients, and mix continuously until a homogeneous emulsion is formed.

### F15. PolyDerm F, A Fragrance Release Topical Gel Delivery System

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Mineral Oil | 4.1 |
| | Petrolatum Jelly | 7.5 |
| B. | Deionized Water | 77.9 |
| | Guar Gum | 1.0 |
| | Benzalkonium Chloride | 0.5 |
| | Glycerol | 5.0 |
| C. | Citricidal | 0.5 |
| D. | Phenethyl Alcohol | 0.5 |

Add a sufficient volume of deionized water, glycerol and Benzalkonium chloride, mix thoroughly, and heat the Part B ingredients at 90° C. until the gum is entirely dissolved. Remove from heat and cool to 65° C. Weight out Part A ingredient (mineral oil, DC-200, DC-245 and petrolatum jelly) and add directly to pre-heated Part B ingredients. Stir in Part C and D ingredient, and mix continuously until a homogeneous emulsion is formed.

F16. SynDerm F, a fragrance release topical delivery system

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Mineral Oil | 4.2 |
| | Petrolatum Jelly | 9.3 |
| B. | Deionized Water | 74.5 |
| | Carboxymethylcellulose | 2.0 |
| | Benzalkonium Chloride | 0.5 |
| | Glycerol | 5.0 |
| C. | Citricidal | 0.5 |
| | Phenethyl Alcohol | 0.5 |

Add a sufficient volume of deionized water, glycerol and Benzalkonium chloride, mix thoroughly, and heat the Part B ingredients at 90° C. until the CMC is entirely dissolved. Remove from heat and cool to 65° C. Weight out Part A ingredient (mineral oil, DC-200, DC-245 and petrolatum jelly) and add directly to pre-heated Part B ingredients. Stir in Part C and D ingredient, and mix continuously until a homogeneous emulsion is formed.

### F17. PolycelluDerm F, a Microcrystalline Cellulose Fragrance Releasing Topical Delivery System

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Mineral Oil | 4.1 |
| | Petrolatum Jelly | 9.3 |
| B. | Deionized Water | 72.2 |
| | Guar gum | 0.5 |
| | Cellulose/cellulose | 3.4 |
| | Benzalkonium Chloride | 0.5 |
| | Glycerol | 5.0 |
| C. | Citricidal | 0.5 |
| D. | Phenethyl Alcohol | 0.5 |

Add a sufficient volume of deionized water, glycerol and Benzalkonium chloride, mix thoroughly, and heat the Part B ingredients at 80° C. until the gum is entirely dissolved. Remove from heat and cool to 65° C. Weight out Part A ingredient (mineral oil, DC-200, DC-245 and petrolatum jelly) and add directly to pre-heated Part B ingredients. Stir in Part C and D ingredient, and mix continuously until a homogeneous emulsion is formed.

### F18. Berri-Seal F, a Natural Emollient Fragrance Releasing Topical Delivery System

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Mineral Oil | 4.1 |
| | Berry Wax | 9.3 |
| B. | Deionized Water | 73.5 |
| | Corn Starch | 3.1 |
| | Glycerol | 5.0 |
| C. | Citricidal | 0.5 |
| D. | Phenethyl Alcohol | 0.5 |

Add a sufficient volume of deionized water, glycerol, mix thoroughly, and heat the Part B ingredients at 90° C. until the starch is entirely dissolved. Remove from heat and cool to 65° C. Weight out Part A ingredient (mineral oil, DC-200, DC-245 and Berry Wax/Soya and Canola oils, EnviroPure306, React-NTI) and add directly to pre-heated Part B ingredients. Stir in Part C and D ingredients, and mix continuously until a homogeneous emulsion is formed.

### F19. EVA/Oil-Seal F: A Natural Emollient Topical Delivery System

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Mineral Oil | 4.1 |
| | Ethylene Vinyl Acetate | 9.3 |
| B. | Deionized Water | 73.5 |
| | Corn Starch | 3.1 |
| | Glycerol | 5.0 |
| C. | Citricidal | 0.5 |
| D. | Phenethyl Alcohol | 0.5 |

Add a sufficient volume of deionized water, glycerol, mix thoroughly, and heat the Part B ingredients at 90° C. until the starch is entirely dissolved. Remove from heat and cool to 65° C. Weight out Part A ingredient (mineral oil, DC-200, DC-245 and binder ethylene vinyl acetate/Soya and Canola oils, EnviroPure301, React-NTI) and add directly to pre-heated Part B ingredients. Stir in Part C and D ingredients, and mix continuously until a homogeneous emulsion is formed.

### F20. Berri/Olive Oil-Derm Seal, a Natural Emollient Fragrance Releasing Topical Delivery System

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Olive Oil | 3.9 |
| | Berry Wax | 9.8 |
| B. | Deionized Water | 72.5 |
| | Corn Starch | 3.3 |
| | Benzalkonium Chloride | 0.5 |
| | Glycerol | 5.0 |
| C. | Citricidal | 0.5 |
| D. | Phenethyl Alcohol | 0.5 |

Add a sufficient volume of deionized water, glycerol, and Benzalkonium chloride, mix thoroughly, and heat the Part B ingredients at 80° C. until the starch is entirely dissolved. Remove from heat and cool to 65° C. Weight out Part A ingredient (olive oil, DC-200, DC-245 and Berry Wax/Olive Oil, EnviroPure310, React-NTI) and add directly to pre-heated Part B ingredients. Stir in Part C and D ingredients, and mix continuously until a homogeneous emulsion is formed.

### F21. Modified Starch-Berri-Derm F, a Natural Emollient Fragrance Releasing Topical Delivery System

| | |
|---|---|
| Ingredient | Wt. % |

| | | |
|---|---|---|
| A. | Poly(dimethylsiloxane) | 0.8 |
| | Decamethylpentanecyclosiloxane | 3.2 |
| | Mineral Oil | 3.8 |
| | Berry Wax | 9.8 |
| B. | Deionized Water | 72.7 |
| | Corn Starch | 3.3 |
| | Benzalkonium Chloride | 0.4 |
| | Glycerol | 5.0 |
| C. | Citricidal | 0.5 |
| D. | Phenethyl Alcohol | 0.5 |

Weigh the Part B starch (PureDent 836, hydrophobically-modified corn starch, Grain Processing Corp., Muscatine, Iowa) ingredient and place in suitable vessel equipped with mixer. Add a sufficient volume of deionized water, glycerol, and Benzalkonium chloride, mix thoroughly, and heat the Part B ingredients at 80° C. until the starch is entirely dissolved. Remove from heat and cool to 65° C. Weight out Part A ingredient (Mineral oil, DC-200, DC-245 and Berry Wax/Soya and Canola Oil, EnviroPure306, React-NTI) and add directly to pre-heated Part B ingredients. Stir in Part C and D ingredients, and mix continuously until a homogeneous emulsion is formed.

In summary, we have described a method for producing stable dispersions of oil droplets in a starch matrix. The process first requires gelatinizing natural starch at a temperature sufficient to dissolve starch in an aqueous solution containing one or more emulsifying agents and, then blending of one or more oils with the gelatinized starch phase at a temperature sufficient to prevent gel formation. The resulting gels (lotions) are greaseless and tack-less have good spreadability, rapid drying, and water- and alcohol resistance. These materials form a protective and occlusive film on skin. Glycerol, a humectant, can be used in the aqueous phase to provide for additional skin moisturization.

Further, the microemulsions prepared by the above process are able to deliver hydrophobic botanical extracts, and may be useful for the delivery of cosmetic and medicinal ingredients. Such hydrocolloid emulsions deliver botanicals with anti-oxidant, anti-aging, and anti-irritant properties. In particular, formulation 9 through 12 were shown to be good topical delivery systems with diverse personal care applications.

It should be readily apparent that Thixogel microemulsions are both easy to formulate and cost-effective. The major ingredients such as cornstarch, vegetable oils, mineral oil, and petrolatum are relatively inexpensive. Since a stable thixotropic microemulsion of starch-in-oil requires only very low levels of an emulsifying agent, the formulator can avoid the use of expensive fatty acid alcohols, fatty acid esters, thickeners, and emulsion stabilizers, that are generally required to produce stable oil-in-water emulsions. Unlike many cosmetic emulsions, Thixogel microemulsions are completely greaseless, and leave no oily residue on the skin. Furthermore, they are completely resistant to alcohol and thus do not wash off when body skin is rinsed or decontaminated with alcohol. This property makes them highly useful to healthcare workers, who can avoid the irritant effects of multiple cycles of water and alcohol washes during the course of their sanitary protocols.

There have been shown and described methods for the preparation of a novel thixotropic microemulsion and numerous skin care formulations. In addition, methods were presented for the preparation of formulations that deliver moisture-activated fragrance release from a starch-oil microemulsion. These and other properties are exemplified in the examples and formulations in the preferred embodiments of the present invention. It is to be understood, that the specific ingredients cited in the above examples are not limited to those alone but can be any of the components that that are generally useful conferring skin moisturization, skin protection, and volatile fragrances generally employed in cosmetic applications and to those familiar with the state of the art in cosmetic formulations. Many changes, modifications, variations and other uses and applications of the subject invention will, however, become apparent to those skilled in the art after considering the specification and the accompanying examples and formulations which disclose the preferred embodiments thereof. All such changes, modifications, variations and other uses and applications which do not depart from the spirit and scope of the invention are deemed to be covered by the invention, which is not to be limited only by the claims which follow.

I claim:

1. A novel method of preparing a greaseless, tack-free oil-in-water (O/W) thixotropic microemulsion and topical formulations derived therefrom that are useful as skin barrier and moisturizing cosmetic lotions, said method comprising the steps of:
i) forming the aqueous phase of said emulsion by mixing together in a suitable vessel: a) about 1% to 10% (W/V) of natural cornstarch or polysaccharide thickeners, b) about 0.1 to 3% (W/V) of a cationic or amphoteric emulsifying surfactant, and c) about 1-10% (V/V) of a humectant, and heating the mixture at between 70° C. and 80° C. to obtain a clarified starch gel; and
ii) forming an oil-in-water microemulsion by adding: c) from 1% to 25% (V/V) of a cosmetically acceptable natural or synthetic oil, d) from about 0.1% to 5% (V/V) of an oil-soluble volatile fragrance, and e) and from 0.1% to 5% of an oil-soluble natural preservative to the above aqueous phase, and heating the mixture of the two phases at between 70° C. and 75° C. with continuous low shear stirring until a uniform and stable emulsion is formed.

2. The method of claim 1, wherein the average particle size distribution of the oil droplets dispersed within the microemulsion is about 0.5 to 1.0 microns, and the average viscosity is about 8,000 to 12,000 cps.

3. The method of claim 1, wherein the carbohydrate rheological modifier is selected from a group of natural starches consisting of food grade corn starch, potato starch and waxy maize corn starches each having a molecular weight of not less than 1-2×10⁶ Daltons.

4. The method of claim 1, wherein the rheological modifier is a high molecular weight polysaccharide selected from a group consisting of guar gum, sodium carboxymethylcellulose, and a microcrystalline cellulose.

5. The method of claim 1, wherein the cosmetically acceptable oil is selected from a group of vegetable oils consisting of oleic acid, linoleic acid, palmitoleic acid, canola oil, linseed oil, cottonseed oil, meadowfoam oil, sea buckthorn soil, soybean oil, olive oil, and berry waxes.

6. The method of claim 1, wherein the synthetic oil is selected from a group consisting of mineral oil, petrolatum, squalane and skin-protecting silicone oils.

7. The method of claim 6, wherein the skin-protecting oils include at least one of dimethicone, decamethyl cyclopentanesiloxane and combinations thereof.

8. The method of claim 1, wherein the volatile oils are selected from a group consisting of an insect repellent oil, a fragrance, an essential oil and an aroma therapy oil.

9. The method of claim 8, wherein the insect repellent oil is DEET.

10. The method of claim 8, wherein the oil-soluble volatile fragrance is phenethyl alcohol.

11. The method of claim 8, wherein the essential oil is sweet orange.

12. The method of claim 1, wherein the cationic emulsifier is selected from a group consisting of benzalkonium chloride, distearyldimonium chloride and combinations thereof, and the amphoteric surfactant is selected from a group consisting of lecithin, phosphatidylcholine, phosphatidylethanolamine and phosphatidylinositol.

13. The method of claim 1, wherein the humectant is glycerol.

14. The method of claim 1, wherein the oil is 1% to 10% perfurorodecalin, an oxygen-carrying oil molecule.

15. The method of claim 1, wherein the oil-soluble volatile preservative is a natural preservative selected from a group consisting of grapefruit seed oil and tea tree oil.

16. The method of claim 1, wherein the oil-soluble volatile fragrance is capable of moisture-activated release from within oil droplets that are encapsulated in a carbohydrate capsule.

17. The method of claim 1, further comprising the steps of preparing a starch-oil composite film from the liquid emulsion and reducing the film to a powder employing a micronizing mill.

18. The method of claim 17, wherein the starch-oil composite film is prepared by air drying the liquid emulsion under ambient temperature.

19. The method of claim 17, wherein the starch-oil composite film is prepared by heating and drying the liquid emulsion using standard spray drying or drum drying procedures known in the art of the film forming industry to obtain films having less than 5% moisture content.

20. The method of claim 17, wherein release of the volatile oils from the dried starch-oil composite film formed on skin is achieved by transepidermal water loss through the skin, by process of ordinary sweating or by direct application of water to the dried films or powders.

21. The method of claim 17, wherein a sequestered fragrance in the starch-oil composite film is stored therein in the form of a sealed liquid emulsion, subsequently dried and milled to a powder, and the powder is stored under low moisture conditions.

22. The method of claim 21, wherein the sequestered fragrance is stored until fragrance release is required and initiated by rehydrating the powder with water moisture to form an instant lotion composition containing at least one of an encapsulated volatile fragrance and another temperature-sensitive ingredient.

23. The method of claim 1, further comprising fabricating a wound dressing from a dried starch film formed by drying a 2:1 dilution of said thixotropic microemulsion comprising from about 1% to about 10% starch, a cationic surfactant, and from about 1% to about 10% oil.
